(19) United States
(12) Plant Patent Application Publication
(10) Pub. No.: US 2024/0381800 P1
(43) Pub. Date: Nov. 14, 2024

(54) CANNABIS PLANT NAMED 'PTC8506V'

(50) ;

(71) Applicant: Phylos Bioscience, Inc., Portland, OR (US)

(72) Inventor: Jared Reynbery, Portland, OR (US)

(73) Assignee: Phylos Bioscience, Inc., Portland, OR (US)

(21) Appl. No.: 18/643,930

(22) Filed: Apr. 23, 2024

(51) Int. Cl. A01H 6/28

(52) U.S. Cl. CPC A01H 6/28
USPC PLT/287

(57) ABSTRACT

A new and distinct variety of Cannabis sativa designated ‘PTC8506V’ as herein described and illustrated, characterized by elevated levels of THCV and THC.

Genus/species: Cannabis sativa.

Variety denomination: ‘PTC8506V’.

### BACKGROUND

Cannabis plants contain over a hundred known cannabinoids, which bind to endogenous endocannabinoid receptors. Varinolic cannabinoids, as known as varins, are a type of cannabinoid compounds having three carbon atoms in their alkyl side chain instead of the five carbon atom alkyl side chains more commonly associated with cannabinoids. Two such varins are tetrahydrocannabivarin (THCV) and cannabidivarin (CBDV), which are homologues of tetrahydrocannabinol (THC) and cannabidiol (CBD), respectively. Each varin has a unique pharmacological profile and distinct molecular targets.

THCV and CBDV have potential benefits across a broad set of applications. Cannabis plants or extracts with high THCV levels, for example, can be used as an agent for anticonvulsant activity, obesity-associated glucose intolerance, appetite suppression, anxiety management for PTSD, diabetic neuropathy, and major neuropathic and pain related pathologies. Another THCV application is that of an appetite suppressing compound. CBDV has been shown to have anti-epileptic and anticonvulsant activity.

Research and development as well as the sale of varin products has been limited due to low commonly occurring levels of varins in Cannabis flower. The ability to produce Cannabis with high varin levels will create a platform for a new Cannabinoid category with differentiated, high margin products in both medical and recreational markets. The present application solves these problems by providing a novel Cannabis plant having high THCV and low THC levels, which is referred to by the variety name ‘PTC8506V’.

### SUMMARY

The new variety of Cannabis sativa was created by sourcing Cannabis plants that produce THCV and THC. Crosses were conducted to elevate further THCV, THC and/or other varins. All breeding, cultivation, and chemotype analyses were conducted in Hillsboro, Oregon. ‘PTC8506V’ resulted from the cross of a high THC pollen receiver plant (‘21GAQ-1271’, unpatented) and a high THCV pollen donor plant (‘21VLP5-1-102’, unpatented). ‘21GAQ-1271’ is a clonal variety with a THC content of 22.2%, and a THCV content below the limit of detection (LOD). ‘21VLP5-1-102’ had a THC content of 1.2%, and THCV content of 5.66%. A clone of ‘21VLP5-1-102’ was crossed onto a clone of ‘21GAQ-1271’ in 2021. 32 of the resulting F1 seed were sown. ‘PTC8506V’ was discovered and selected as a single plant from this F1 seed and was subsequently propagated clonally in Hillsboro, OR. ‘PTC8506V’ was asexually reproduced by apical and sub-apical semi-herbaceous stem cuttings in Hillsboro, Oregon for eight cycles. Asexual reproduction has demonstrated that ‘PTC8506V’ reproduces true to type with all of the characteristics, as herein described, and firmly fixed and retained through successive generations of asexual propagation.

The new variety is named ‘PTC8506V’ and is characterized as having elevated levels of THCV and THC.

### BRIEF DESCRIPTION OF THE PHOTOGRAPH

FIG. 1 is an image of a flowering plant of ‘PTC8506V’ growing in Hillsboro, Oregon.

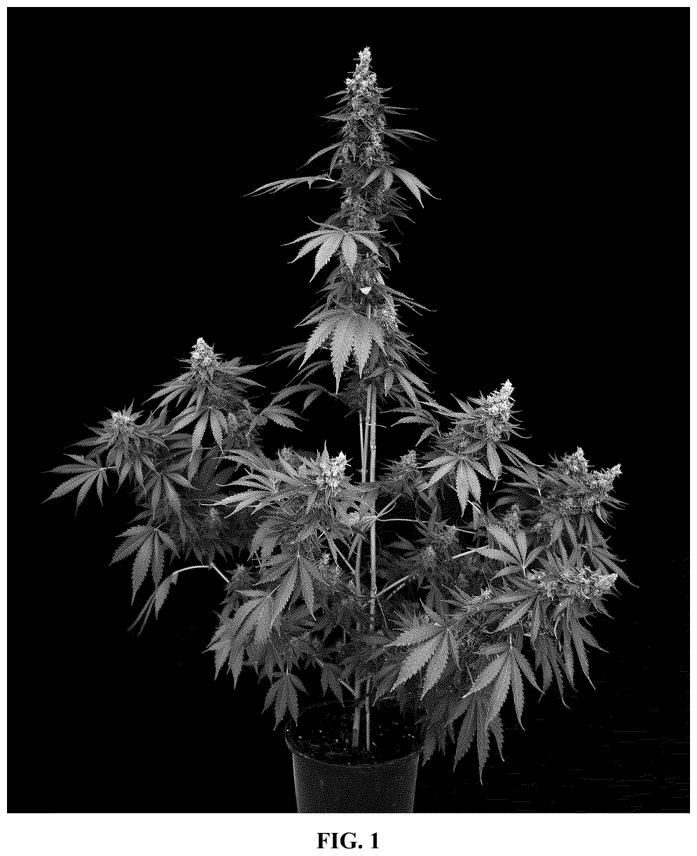

### DETAILED BOTANICAL DESCRIPTION

The following is a detailed description of the new Cannabis variety ‘PTC8506V’.

All breeding, cultivation, and chemotype analyses were conducted in Hillsboro, Oregon.

Cannabis plants having elevated THCV or THC levels were sourced and obtained, and crosses were conducted to produce new varieties to further elevate THCV and THC levels. The plants were crossed to produce new varieties having elevated THCV and THC levels. Plant variety ‘PTC8506V’ was selected and asexually reproduced.

Table 1 describes the general characteristics of ‘PTC8506V’ at 55 days after lighting was changed to 12 hours on/12 hours off.

TABLE 1

| | |
|---|---|
| Plant life forms | An herbaceous plant (herb) |
| Plant growth habit | An upright, tap-rooted annual |
| | plant, forming fibrous roots when |
| | asexually propagated |
| Plant origin | A controlled cross between pollen |
| | acceptor (‘21GAQ-1271’) and pollen |
| | donor plant (‘21VLP5-1-102’). |
| Plant propagation | Asexually propagated by apical |
| | and sub-apical semi-herbaceous |
| | stem cuttings |
| Propagation Ease | Easy |
| Plant shape | Simple erect |
| Height | 117 cm to 121 cm |
| Width | 87 cm to 101 cm |
| Time to harvest | 10-12 weeks |
| Resistance to pests or diseases | Unknown |
| GMO? | No |

Table 2 describes the leaf/foliage of ‘PCT8506V’. Color codes are hex color codes.

TABLE 2

| | |
|---|---|
| Leaf Arrangement | Alternate |
| Leaflet Type | Palmately compound |
| Leaflet Structure | Serrated margins, lightly acicular |
| | to lanceolate leaflets, tapering |
| | to an acuminate apex |
| Leaflet Margins | Jaggedly serrate with each tooth |
| | apex angled toward leaflet apex |
| Leaf hairs | Present on both upper and |
| | lower surfaces |
| Leaf length with petiole at | 18.5 cm to 23 cm |
| maturity | |
| Petiole length at maturity | 6 cm to 9.5 cm |
| Petiole diameter at maturity | 1.9 mm to 2.5 mm |
| Petiole color | #6B4247 |
| Anthocyanin color and intensity | #F9CCC5. Medium intensity in |
| in Petioles | vegetative stage; stronger in |
| | flowering stage |
| Stipule length at maturity | 6.2 mm to 6.8 mm |
| Stipule shape | Linear with slight curvaceous growth |
| Stipule color | #3C4D03 |
| No. of leaflets | 3 to 7 |
| Middle largest (longest) leaflet | 12 cm to 17.5 cm |
| length | |
| Middle largest (longest) leaflet | 2.5 cm to 3.1 cm |
| width | |
| Middle largest (longest) leaflet | 3.8 to 4.8 |
| length/width ratio | |
| No. teeth of middle leaflet | 28 to 34 |
| Leaf (upper side-adaxial) color | #35530A |
| Leaf (lower side-abaxial) color | #35530A |
| Leaf glossiness | Matte |
| Vein/midrib shape | Obliquely continuous |
| | throughout leaflet |
| Vein/midrib color | #E6DAA6 |
| Aroma | Earthy notes of pine, fuel, |
| | and a slight fruitiness |

Table 3 describes the stem of ‘PTC8506V’.

TABLE 3

| | |
|---|---|
| Stem shape | Hollow, ribbed, texture |
| Stem length | 117 cm to 121 cm |
| Stem strength | Strong and upright |
| Stem diameter at base | 13.6 mm to 14.5 mm |
| Stem color | #373E02 |
| Depth of main stem ribs/grooves | Shallow |
| Internode length | 3 cm to 9.5 cm |

Table 4 describes the inflorescence of ‘PTC8506V’.

TABLE 4

| | |
|---|---|
| Inflorescence type | Elongated compound cymes |
| Flowering (blooming) habit | Natural flowering season is Autumn. |
| Proportion of female plants | 100% pistillate |
| Inflorescence position | Terminal and axillary |
| Number of inflorescences produced per plant | 100 to 150 |
| Flower arrangement | Cymose |
| Length of inflorescences | 3 cm to 3.8 cm |
| Numbers of flowers per plant | Hundreds to thousands flowers per plant, |
| | multitudinous, congested with high |
| | concentrations on bushier wide inflorescence |
| Flower shape | Urceolate |
| Flower (individual pistillate) length | 5.1 mm to 5.3 mm |
| Flower (individual pistillate) depth | 2 mm |
| Flower (compound cyme) diameter | 5.5 cm to 8 cm |
| Corolla | No defined corolla |
| Corolla color | N/A |
| Bract shape | Urceolate |
| Bract color | #4B5D16 |
| Typical bract numbers | 3 to 200 bracts per inflorescence. |
| Bract length | 3 mm to 5mm |
| Bract width | 2.3 mm to 2.7 mm |
| Bract apex and base description | Bracts have a rounded base and caudate |
| | apex. |
| Bract Texture | Bract texture is smooth and covered with |
| | sticky trichomes. |
| Bracteole number, shape, length, width, apex, | Bracteoles are not present |
| margin, base descriptors, texture, and color | |
| Calyx shape | No defined calyx |
| Calyx color | N/A |
| Pistil number per flower | One |
| Pistil color | Pistil consists of the stigma, style, and ovary |
| | and cannot be given a single color. |
| Stigma shape | Acute |
| Number of stigmas per flower | Two |
| Stigma length | 4.6 mm to 6.5 mm |
| Stigma color | #373E02 |
| Trichome shape | Capitate-stalked glandular |
| Trichome color | #F1E7E2 |
| Terminal inflorescence shape | Oblong |
| Terminal inflorescence color | #B7E1A1 |
| Terminal inflorescence length | 12.5 cm to 14 cm |
| Terminal inflorescence width | 5 cm to 6.5 cm |
| Pedicel | Absent |
| Peduncle length | 6.8 mm |
| Peduncle diameter | 4.1 mm |
| Peduncle texture | Lightly ribbed |
| Peduncle color | #9BB53C |
| Staminate shape | No staminate flowers produced naturally; |
| | however, male flower (staminate) can be |
| | induced with stress or ethylene-blocking |
| | compounds |
| Sepal color | N/A |
| Pollen description | N/A |
| Seed shape | Striped, smooth and globular |
| Seed size | 1.6 mm to 2.3 mm |
| Petal description | Apetalous |

Table 5 describes other characteristics of ‘PTC8506V’

TABLE 5

| | |
|---|---|
| Time period and condition of | 8 to 10 weeks |
| flowering/blooming | |
| Proportion of hermaphrodite | Lower (<5%) in clones |
| plants | |
| Hardiness of plant | Hardy to tolerate of warm and dry |
| | conditions without showing stress. |
| | Not tolerant to soil oversaturated |
| | with water for long periods |

Chemical analyses were conducted using High Performance Liquid Chromatography to detect the cannabinoids described herein. Table 6 describes a chemotype analysis of ‘PTC8506V’, which was chemotyped by taking a sample from the main cola at day 56 after initiation of the 12:12 light cycle, which initiates flowering. Trichomes were 70% cloudy and 5% amber, indicative of full maturity at sampling.

TABLE 6

| Cannabinoid and other chemicals | % wt |
|---|---|
| Total THC | 4.613 |
| Total THCV | 11.067 |
| Total CBG | 0.047 |
| Total CBD | <LOD |
| Total CBC | <LOD |
| Total CBGV | <LOD |
| Total CBDV | <LOD |
| alpha-Pinene | 0.057 |
| Camphene | 0.011 |
| beta-Myrcene | 1.557 |
| beta-Pinene | 0.098 |
| delta-3-Carene | <LOQ |
| alpha-terpinene | <LOQ |
| D-Limonene | 0.211 |
| beta-Ocimene | 0.275 |
| p-Cymene | <LOQ |
| Eucalyptol | <LOQ |
| gamma-Terpinene | <LOQ |
| Terpinolene | <LOQ |
| Linalool | 0.003 |
| Isopulegol | <LOQ |
| Geraniol | <LOQ |
| beta-Caryophyllene | 0.05 |
| alpha-Humulene | 0.0227 |
| Nerolidol 1 | <LOQ |
| Nerolidol 2 | 0.011 |
| Guaiol | <LOQ |
| Caryophyllene Oxide | 0.011 |
| alpha-Bisabolol | 0.026 |
| Total Terpenes | 2.315 |

What is claimed is:

1. A new and distinct variety of Cannabis sativa designated ‘PTC8506V’ as herein described and illustrated.
